SEQUENCE LISTING (24 sequences)

SEQ ID NO: 1 - DNA, 10 bp, Mus musculus
rycayrtgwy

SEQ ID NO: 2 - DNA, 15 bp, Mus musculus
  misc_feature (7)..(8): n is a, c, g, or t
rgkwcdnnkhgttmy

SEQ ID NO: 3 - DNA, 15 bp, Mus musculus
  misc_feature (9)..(9): n is a, c, g, or t
  misc_feature (13)..(13): n is a, c, g, or t
gdwwcdtynhgwnhy

SEQ ID NO: 4 - DNA, 11 bp, Mus musculus
arrkrggvgkk

SEQ ID NO: 5 - DNA, 16 bp, Mus musculus
wrkrrsasmrdrdkbw

SEQ ID NO: 6 - DNA, 15 bp, Mus musculus
  misc_feature (2)..(2): n is a, c, g, or t
wndrkrkcrwrgkkb

SEQ ID NO: 7 - DNA, 20 bp, Artificial Sequence
  Primer
caagcagaagacggcatagc

SEQ ID NO: 8 - DNA, 33 bp, Artificial Sequence
  Primer
acactctttccctacacgacgctcttccgatct

SEQ ID NO: 9 - DNA, 33 bp, Artificial Sequence
  Library Adaptor
gatcggaagagctcgtatgccgtcttctgcttg

SEQ ID NO: 10 - DNA, 15 bp, Mus musculus
  misc_feature (1)..(1): n is a, c, g, or t
  misc_feature (3)..(4): n is a, c, g, or t
  misc_feature (7)..(11): n is a, c, g, or t
  misc_feature (14)..(15): n is a, c, g, or t
nbnnddnnnnndknn

SEQ ID NO: 11 - DNA, 10 bp, Mus musculus
  misc_feature (1)..(3): n is a, c, g, or t
  misc_feature (10)..(10): n is a, c, g, or t
nnngkrgghn

SEQ ID NO: 12 - DNA, 11 bp, Mus musculus
  misc_feature (1)..(1): n is a, c, g, or t
  misc_feature (8)..(8): n is a, c, g, or t
ngddvdwnmcm

SEQ ID NO: 13 - DNA, 10 bp, Mus musculus
  misc_feature (1)..(2): n is a, c, g, or t
nnhhrrhdac

SEQ ID NO: 14 - DNA, 15 bp, Mus musculus
  misc_feature (2)..(3): n is a, c, g, or t
  misc_feature (6)..(6): n is a, c, g, or t
  misc_feature (10)..(10): n is a, c, g, or t
hnndhnhvsnmcvrb

SEQ ID NO: 15 - DNA, 11 bp, Drosophila melanogaster
rtkrysymmbv

SEQ ID NO: 16 - DNA, 11 bp, Drosophila melanogaster
  misc_feature (6)..(6): n is a, c, g, or t
  misc_feature (11)..(11): n is a, c, g, or t
taaycnghbhn

SEQ ID NO: 17 - DNA, 11 bp, Drosophila melanogaster
  misc_feature (9)..(9): n is a, c, g, or t
  misc_feature (11)..(11): n is a, c, g, or t
hhhcmydbnhn

SEQ ID NO: 18 - DNA, 14 bp, Drosophila melanogaster
svcacmygrsacts

SEQ ID NO: 19 - DNA, 11 bp, Homo sapiens
  misc_feature (11)..(11): n is a, c, g, or t
hdyagtrkkdn

SEQ ID NO: 20 - DNA, 21 bp, Homo sapiens
  misc_feature (2)..(2): n is a, c, g, or t
  misc_feature (11)..(12): n is a, c, g, or t
  misc_feature (15)..(15): n is a, c, g, or t
  misc_feature (17)..(18): n is a, c, g, or t
  misc_feature (21)..(21): n is a, c, g, or t
vnmmhrtmhmnnmmnvnnvhn

SEQ ID NO: 21 - DNA, 15 bp, Homo sapiens
  misc_feature (1)..(1): n is a, c, g, or t
  misc_feature (3)..(3): n is a, c, g, or t
  misc_feature (7)..(7): n is a, c, g, or t
  misc_feature (9)..(9): n is a, c, g, or t
  misc_feature (13)..(13): n is a, c, g, or t
  misc_feature (15)..(15): n is a, c, g, or t
nhnmhwndnrdgndn

SEQ ID NO: 22 - DNA, 15 bp, Homo sapiens
  misc_feature (1)..(1): n is a, c, g, or t
  misc_feature (7)..(7): n is a, c, g, or t
  misc_feature (10)..(10): n is a, c, g, or t
ndvvyvnybndbwky

SEQ ID NO: 23 - DNA, 11 bp, Mus musculus
yrsgkgggbrk

SEQ ID NO: 24 - DNA, 10 bp, Mus musculus
trygkrgghv

(19) United States
(12) Patent Application Publication
(10) Pub. No.: US 2019/0091689 A1
(43) Pub. Date: Mar. 28, 2019

(54) Microfluidic Device and Method for Isolation of Nucleic Acid

(71) Applicant: ECOLE POLYTECHNIQUE FEDERALE DE LAUSANNE (EPFL), Lausanne (CH)

(72) Inventors: Alina Isakova, Morges (CH); Bart Deplancke, Cugy (CH)

(21) Appl. No.: 15/518,264

(22) PCT Filed: Oct. 19, 2015
(86) PCT No.: PCT/IB2015/058032

§ 371 Date: Apr. 11, 2017

(30) Foreign Application Priority Data
Oct. 17, 2014 (IB) PCT/IB2014/065418

(51) Int. Cl. B01L 3/00; C12N 15/115; C40B 60/02; C40B 60/12

(52) U.S. Cl. CPC B01L 3/502761; C12N 15/115; C40B 60/02; B01L 2400/0655; B01L 2300/0864; B01L 2300/0877; C40B 60/12

(57) ABSTRACT

The present invention concerns a microfluidic device for mechanically induced trapping of molecular interactions comprising at least a first unit cell and a second unit cell, each unit cell comprising—a membrane chamber comprising a membrane, —a flow channel crossing the membrane chamber and having an inlet and an outlet, and the flow channel crossing the first unit cell being different from the flow channel crossing the second unit cell. Another object of the invention is a method for isolation of specifically bound nucleic acids to target molecules on said microfluidic device followed by its recovery and identification.

### CROSS-REFERENCE TO RELATED APPLICATIONS

This application claims the benefit of international patent application PCT/IB2014/065418 filed Oct. 17, 2014 the entire contents of which are incorporated herein by reference.

### REFERENCE TO A SEQUENCE LISTING

The instant application contains a Sequence Listing which has been submitted electronically in ASCII format and is hereby incorporated by reference in its entirety. The copy of the Sequence Listing, created on Aug. 21, 2018, is named P2826US00_SeqList_ST25.txt and is 6,661 bytes in size. This application contains a partial sequence list in Table 5.

### FIELD OF THE INVENTION

The present invention generally relates to the identification of nucleic acids specifically bound to organic targets. More specifically the present invention relates to: 1) microfluidic devices which are capable of selective isolation and purification of nucleic acids specifically bound to protein targets; 2) method for preparation, on-chip processing and recovery of nucleic acids that are specifically bound to protein targets and are directly compatible with high-throughput sequencing.

### BACKGROUND OF THE INVENTION

Identification of nucleic acids specifically bound to organic targets possesses an important niche of academic and industrial research. Discovery and development of non-traditional drugs based on innate, non-toxic and degradable organic compounds, such as nucleic acids, is an actively evolving branch of pharmaceutical and biotechnology industry. At the same time, knowing which nucleic acid sequences in a genome are recognized by which proteins is crucial for understanding the gene regulatory networks underlying various biological processes and still remains an important challenge of fundamental science. To this end, both, academia and industry are constantly looking for innovative technologies that aim to decrease costs and increase efficiency of isolation and identification of nucleic acid ligands selectively bound to specific biological targets.

Currently this type of screening is typically done through the use of one of the two technologies: SELEX (Systematic Evolution of Ligands by Exponential Enrichment) or microarrays. Despite the fact that several nucleic acid ligands have been identified through these technologies, the tedious procedure associated with screens as well as the cost of the screen remain a big issue for the massive integration of these standard technologies into any academic or drug developmental toolkit.

Here we present a novel technology, MITOMI-seq, aimed to increase efficiency and throughput as well as to enhance the quality of such screens. MITOMI-seq is based on two innovations:

1. Integrative parallel microfluidic platform for isolation of nucleic acids specifically bound to protein targets. The perform selection assays on this platform only minute amounts of biological material are required.
2. Method for rapid unbiased single-step on-chip selection of nucleic acid specifically bound to protein targets from a pool of randomized DNA or RNA.

Knowing which sites in a genome are recognized by which Transcription Factors (TFs) is crucial for understanding the gene regulatory networks underlying various biological processes. The characterization of the binding preferences of TFs and TF complexes remains an important challenge of molecular biology. However, there are still a large number of factors for which the DNA binding sites are not yet known. Moreover, none of the existing techniques, able to detect TF-DNA interactions, has been used to explore the comprehensive mapping of DNA binding specificities of TF heterodimers or even larger complexes.

In this invention we aimed to tackle the challenge of robust identification of nucleic acid sequences specifically bound to organic targets within a wide affinity range. We demonstrated the power of this novel microfluidics-based technology that exploits the power of next-generation sequencing, by characterizing the DNA binding preferences of a variety of TFs and TF complexes.

### Microfluidics

Microfluidics is the science and technology of manipulating fluids within the networks of micro channels. Microfluidic devices offer an ability to work with volumes that range from micro- to femtoliters and a possibility of parallel sample operation. Within the last ten years, the number of biological applications that involve microfluidics has significantly increased, mostly due to the development of novel micro components and techniques for introducing, mixing (Hong and Quake, 2003; Weibel et al., 2005), pumping (Laser and Santiago, 2004) and storing fluids in microfluidic channels. Currently, microfluidic devices hold a great promise of integrating an entire laboratory onto a single chip (i.e. lab-on-a-chip device).

Until the 1980s, when micro molding of polymers was introduced, microfluidic devices were mainly fabricated on silicon and glass substrates. This required specialized facilities, time and cost investments. And still, devices fabricated of glass or silicon are mostly inutile for analyses of biological samples in solutions. Silicon, in particular, is expensive, and opaque to visible and ultraviolet light, so cannot be used with conventional optical methods of sample detection. The introduction of polymer molding, or so-called soft lithography, enabled fabrication of cheap microfluidic devices that were found to be compatible with multiple biological assays and were quickly adapted by academic laboratories. Currently, the fabrication of microfluidic devices tailored toward biological applications is predominantly based on micro molding of polydimethylsiloxane (PDMS). PDMS is transparent, biocompatible and permeable to gas. Taken together, these unique properties created a strong interest of the scientific community in using this material for microfluidics-based devices. It was also established that multiple patterned layers of PDMS could be easily bonded together, creating complex integrative microfluidic circuits (Unger, 2000).

A good example of an integrative microfluidic device for biological assays is the MITOMI (Mechanically Induced Trapping of Molecular Interactions) platform, which was originally developed to characterize protein-DNA and protein-protein interactions. The physical trapping of molecular interactions on a microchip was one of the foremost technologies that could carefully isolate and quantify molecular complexes. This allowed MITOMI to detect molecular interactions at an unprecedented resolution. It was first applied to study the energy of TF-DNA interactions (Maerkl and Quake, 2007) and later was expanded to measure molecular interaction kinetics (Geertz et al., 2012) and to perform immunoassays on a chip (Garcia-Cordero and Maerkl, 2014).

### RELATED PATENTS/PATENT APPLICATIONS

1. WO_2010019969_A1 Device for rapid identification of nucleic acids for binding to specific chemical targets
Innovative step compared to this patent application:
Usage of “button” membrane that allows reducing the number of selection cycles to one.
Design of the device
Method for a single-step selection of nucleic acids
   2. U.S. Pat. No. 7,143,785 B2 (US 20100154890) Microfluidic Large scale integration Innovative step compared to this patent:
Method to recover bound (“trapped”) nucleic acids from the micro chip
Usage of random nucleic acid libraries compatible with HT sequencing
Presence of cross talk-devoid micro chambers accessible independently

### 3. WO_2007117346 (US 20070248971)

Programming microfluidic devices with molecular information and

### US 20070224617 A1

Mechanically induced trapping of molecular interactions
Innovative step compared to this patent application:
Method to recover bound (“trapped”) nucleic acids from the micro chip
Presence of cross talk-devoid micro chambers accessible independently
Usage of randomized nucleic acid libraries compatible with HT sequencing

### SUMMARY OF THE INVENTION

The present invention concerns a microfluidic device according to claim 1, a dispenser according to claim 6, a method for isolation of specifically bound nucleic acids to target molecules according to claim 10 and uses of said method according to claims 21 to 24.

Other advantages are provided by the features of the dependent claims.

### Industrial Applications of the MITOMI-Seq of the Present Invention:

### 1. Identification of Genomic Targets of Transcription Factors.

An immediate and straightforward application of MITOMI-seq is a comprehensive identification of DNA sites bound by transcription factors monomers and dimers. As described in the methods, the technology could already be applied to robustly identify the genomic targets of multiple proteins at a time in a high-throughput and time-effective manner. This could be particularly interesting for academic research focused on any cellular process implicated in health or disease. It has been widely acknowledged that understanding of biological mechanisms behind any physiological or pathological condition, such as cancer, stem cell renewal or cellular differentiation, goes down to the identification of key transcriptional regulators and its respective DNA targets. MITOMI-seq has a potential to become an indispensable tool for screening the protein-DNA, DNA-RNA and protein-RNA interactions overcoming previously available technologies such as DNA microarrays, EMSA, DNA pull-down in respective cost and throughput.

Another field of application for MITOMI-seq is a clinical research. There MITOMI-seq could be applied to rapidly identify the influence of drugs or modifications on the ability of DNA-binding proteins to recognize their potential target sites. The direct applications of MITOMI-seq could be: drug screening and evaluation.

### 2. Aptamer Screening

Another application of MITOMI-seq could be an aptamer screening.

Aptamers are a class of molecules with a great potential to rival poly- and monoclonal antibodies in therapeutic, diagnostic, analytical as well as basic research applications. Despite the fact that the aptamer technology has been known already for a decade, the identification of novel aptamers specific to the target still remains tedious and cost ineffective. Typically, SELEX technology is used for this purpose. However, as we already showed, MITOMI-seq proposes a robust, cost- and time-effective alternative to standard methods. Particularly, using MITOMI-seq one can perform de novo identification of aptamers specific to a target in parallel and rapid fashion using minute amounts of biological material.

### 3. Single-Cell Analysis

MITOMI-seq holds a great promise to be applied in a rapidly evolving and medically relevant single-cell analysis technologies. MITOMI-seq is a sensitive technique that requires very small amounts of starting material and each step of it could be tightly controlled through the time course of the screen. These properties could be particularly useful when analyzing the biological interactions on a single cell level. Taking into account the fact that MITOMI-seq, similarly to the available cutting-edge technologies aiming for various single-cell analyses, is implemented on a microfluidic device, it could be potentially integrated in other more sophisticated devices.

### Accomplishments of the Invention:

Designed and fabricated a cross talk-devoid micro device consisting of multiple connected units that can be tightly controlled and are accessible independently. The chip allows to perform a robust on-chip selection of TF binding sequences from a size-unbiased pool of randomized DNA using minimal amounts of input biological material.
Designed random DNA libraries that are compatible with HT sequencing and with on-chip TF binding assays.
Designed and implemented a method for rapid isolation of specifically bond DNA to small amount of immobilized protein (target) with its subsequent recovery from the micro device.

### DESCRIPTION OF THE DRAWINGS

The above object, features and other advantages of the present invention will be best understood from the following detailed description in conjunction with the accompanying drawings, in which:

FIG. 1 illustrates an exemplary 64-unit micro device according to the present invention, where FIG. 1A shows a 64-unit chip design in which dark grey and light grey colors denote flow and control layers respectively; FIG. 1B shows that each unit of the device can be accessed independently through an individual flow inlet and an outlet but can also be connected with the other units; FIG. 1C shows that switching between individual and common access modes is done through the use of control valves; and FIG. 1D shows that each device consists of or includes a PDMS chip (approximately 2×5 cm, for example) and an epoxydized glass slide;

FIG. 2 is a schematic representation of MITOMI-seq procedure, where FIG. 2(1) shows a mixture of nucleic acids and protein introduced into the unit of the microfluidic device, protein is immobilized on the surface of the device with antibody; in FIG. 2(2) the system is incubated for one hour to allow its equilibration and complex assembly; in FIG. 2(3) newly formed complexes are trapped under a flexible PDMS membrane and unbound molecules as well as molecular complexes are washed away; and in FIG. 2(4) proteins are disrupted and selectively bound nucleic acids are collected from the device;

FIG. 3 shows chip fabrication according to the present invention;

FIG. 4 shows a mask fabrication process according to the present invention;

FIG. 5 shows a design of two PDMS “passive” dispensers (FIGS. 5A and 5B), an external source of compressed pressure is connected to the inlet of the device and is equally distributed between 64 outlets;

FIG. 6 illustrates extended DNA library design and construction according to the present invention;

FIG. 7 shows a MITOMI-seq procedure according to the present invention, where FIG. 7A shows a snapshot of three units of the microfluidic device, each unit is isolated from others using the microvalves and has an individual inlet and an outlet as illustrated by the independent passage of color dyes; FIG. 7B shows a MITOMI-seq procedure, Bait TF, target dsDNA and a non-specific competitor poly-dIdC are mixed and loaded in one chamber of the microfluidic device, the mixtures are incubated on-chip for 40 min. then, bound DNA is eluted from all the units of the device simultaneously and collected in one tube, recovered DNA is amplified and sequenced on a HiSeq instrument; on the left of FIG. 7B there is a schematic representation of three individual chambers, and on the right there is corresponding snapshots of an individual chamber taken before and after mechanical trapping;

FIG. 8 shows motifs identified by MITOMI-seq for mouse TFs;

FIG. 9 shows motifs identified by MITOMI-seq for fly TFs;

FIG. 10 shows motifs identified by MITOMI-seq for human TFs;

FIG. 11 shows JASPAR, HT-SELEX and MITOMI-seq motif occurrence in ChIP-seq peaks of NFKB1 derived from human lymphoblastoid cell line (GEO:GSM935527); and

FIG. 12 shows pMARE vector maps.

### DETAILED DESCRIPTION OF THE INVENTION

### MITOMI (Mechanically Induced Trapping of Molecular Interactions) Followed by HT (High Throughput) Sequencing (MITOMI-Seq)

We first thought of using original MITOMI devices and an established protocol to perform an on-chip selection assay. Initial experiments revealed however that MITOMI devices suffered from a small and uncontrolled carry-over between neighboring units. Such a cross talk between units is typically not a problem for standard MITOMI applications that require a basic fluorescence-based read out. However, if one wants to recover bound DNA material from the device and subsequently analyze it with an extremely sensitive method like HT-sequencing, even small amounts of cross-contamination between samples may possibly skew data interpretation. Therefore there is a need for a device that can perform mechanically induced trapping of interactions but also allows a controlled isolation of individual units to be one of the key components of a successful on-chip selection procedure.

Thus, we decided to develop a cross talk-devoid device as part of an assay that would allow us to perform a robust on-chip selection of TF binding sequences from a size-unbiased pool of randomized DNA requiring minimal amounts of biological material.

### Microfluidic Device for Parallel Isolation of Specifically Bound Nucleic Acids to Protein Targets

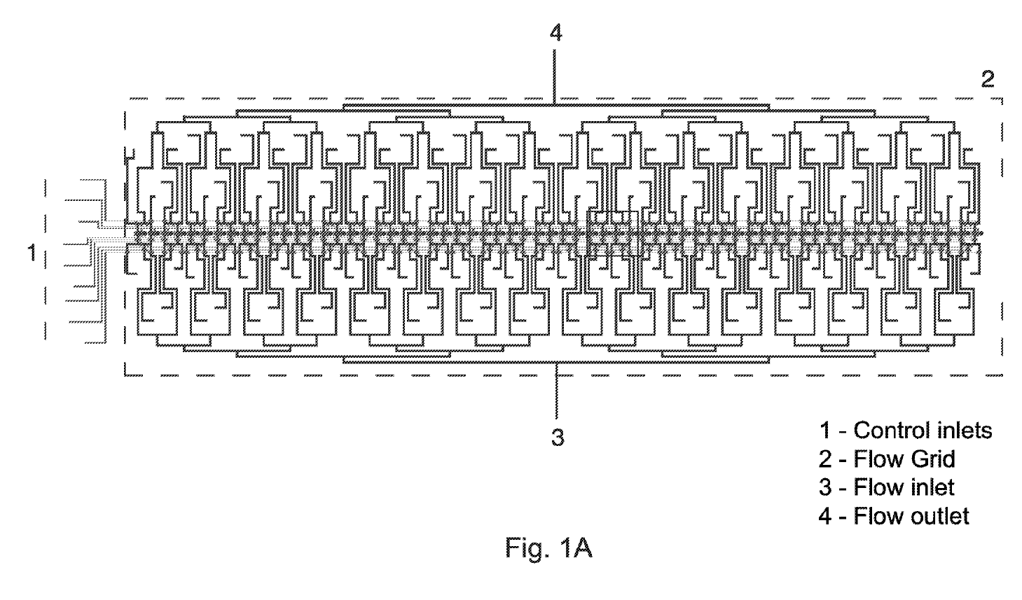

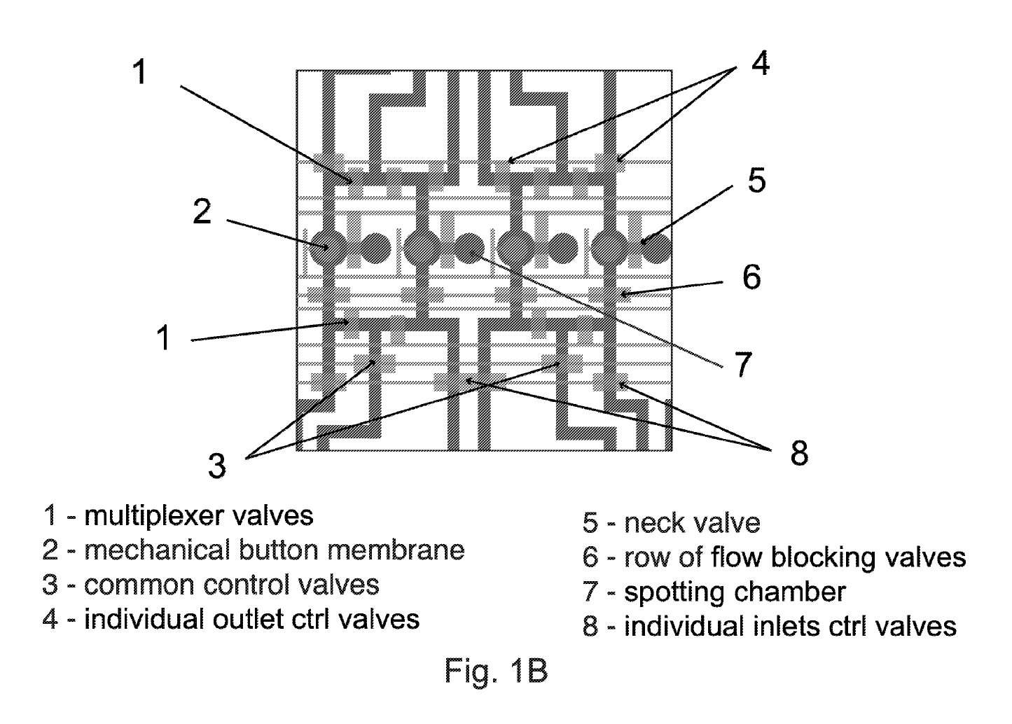

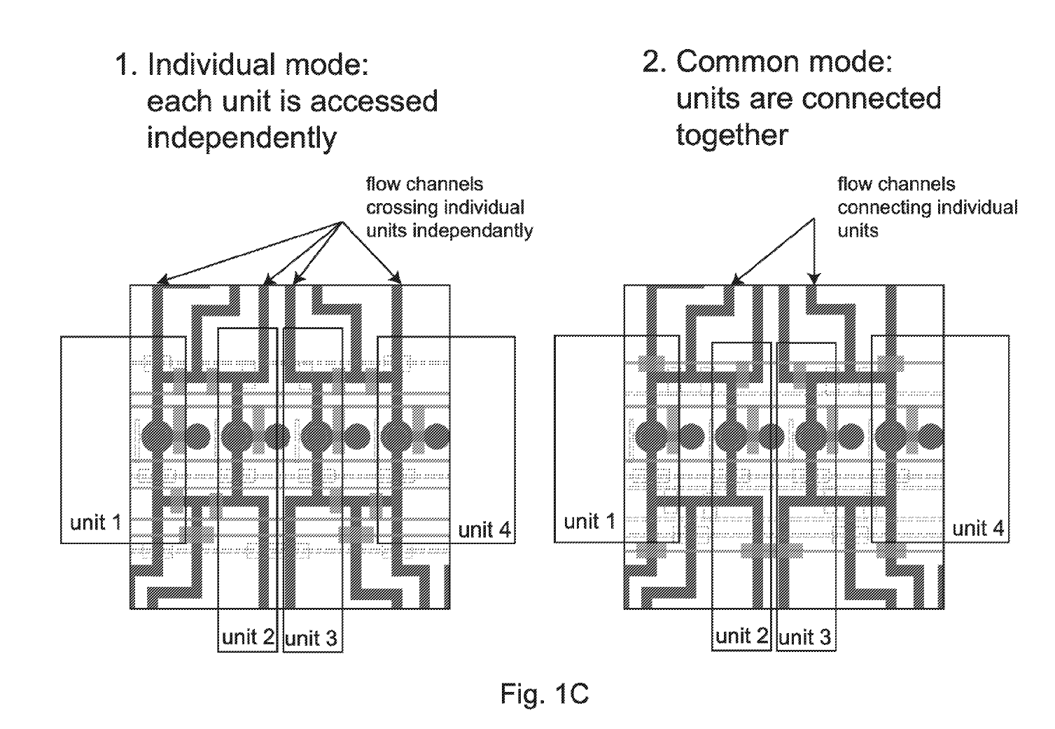

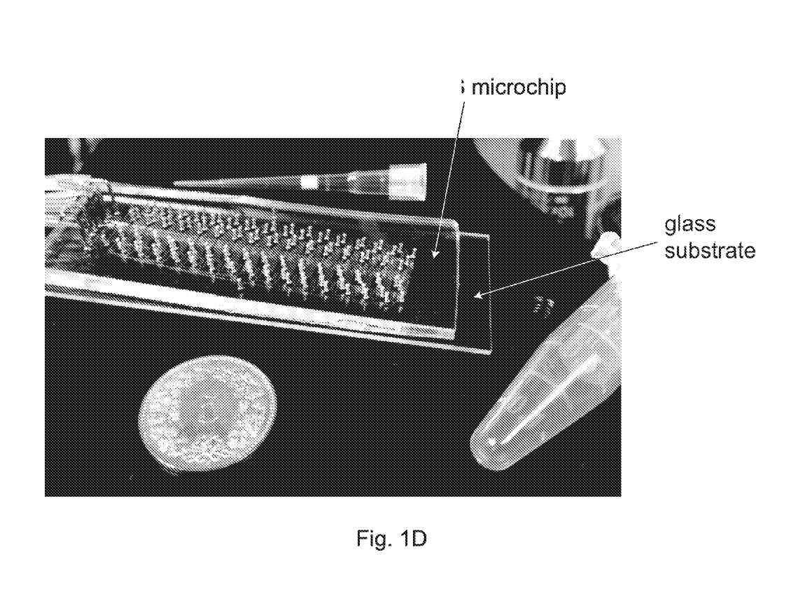

We first designed a micro device that accommodates all the desired features in CleWin software. For convenience we restricted the size of the device to fit a standard 75 by 25 mm glass substrate compatible with an available fluorescent scanner. We designed our device to contain 64 units and set the diameter of the reaction chamber within each unit to 300 μm (FIG. 1). We fabricated the device using two-layer soft lithography at the Center of Micro and Nano Technology at EPFL.

FIG. 1 illustrates an exemplary 64-unit micro device according to the present invention, where FIG. 1A shows a 64-unit chip design in which dark grey and light grey colors denote flow and control layers respectively. FIG. 1B shows that each unit of the device can be accessed independently through an individual flow inlet and an outlet but can also be connected with the other units. FIG. 1C shows that switching between individual and common access modes is done through the use of control valves. FIG. 1D shows that each device consists of or includes a PDMS chip (approximately 2×5 cm, for example) and an epoxydized glass slide;

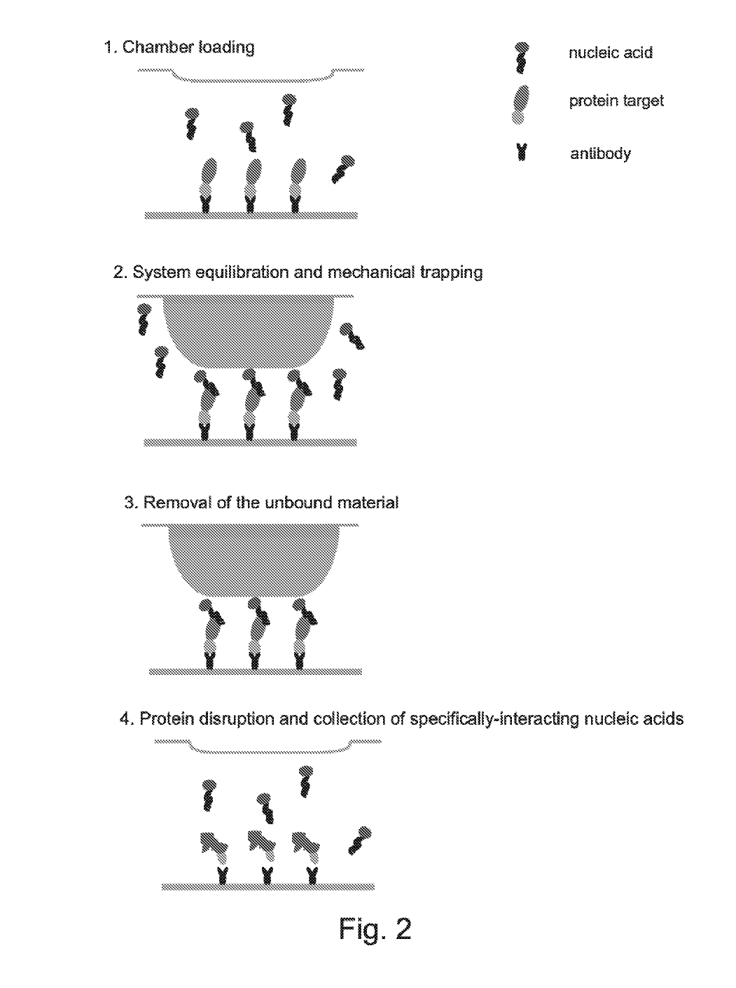

FIG. 1 shows an embodiment of a microfluidic device according to the invention. The device consists of or includes two parts: a PDMS microchip and a substrate (FIG. 1D). A PDMS chip consists of a flow and a control layers which are tightly bonded together. The flow layer is a network of channels and cells, which are tightly controlled by a system of control valves. These valves are essentially thin PDMS membranes that could be deflected by hydraulic forces. Thereby, all manipulations of molecular complexes are performed on the surface of the substrate, in the flow layer of the device. To control the pressure of circulating fluids, the device can be connected to external mechanical or solenoid valves. The mechanical trapping of molecular complexes is done through capping of a circular area with a button valve in each unit cell of the device. FIG. 1B is an enlargement of a part of the microfluidic device. This figure shows four unit cells. Each unit cell comprises:
a membrane chamber, that is crossed by a flow channel that could be operated independently of other unit cells of the device (individual mode, FIG. 1C-1) but could also be connected to other unit cells (common mode, FIG. 1C-2);
a spotting chamber.

FIGS. 1C-1 and 1C-2 illustrate the use of unit cells in an individual and common mode respectively. In individual mode the direction of the flow is controlled by a first set of components: multiplexer valves, neck valves and common control valves, that allow fluid to enter through an individual inlet and exits through an individual outlet. In common mode unit cells are controlled by a second set of components: individual inlets control valves, neck valves and individual outlet control valves, that connect units together and provide a simultaneous accessed to all the units through flow inlet.

FIG. 2 is a schematic representation of MITOMI-seq procedure, where FIG. 2(1) shows a mixture of nucleic acids and protein introduced into the unit of the microfluidic device, protein is immobilized on the surface of the device with antibody. In FIG. 2(2) the system is incubated for one hour to allow its equilibration and complex assembly. In FIG. 2(3) newly formed complexes are trapped under a flexible PDMS membrane and unbound molecules as well as molecular complexes are washed away. In FIG. 2(4) proteins are disrupted and selectively bound nucleic acids are collected from the device.

### Chip Fabrication

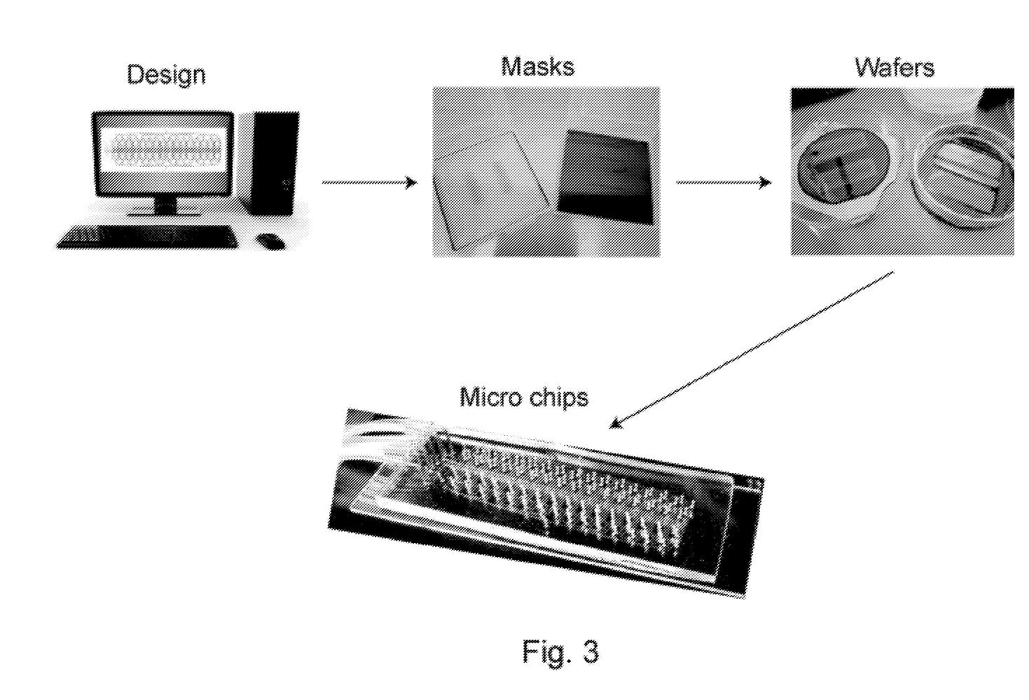

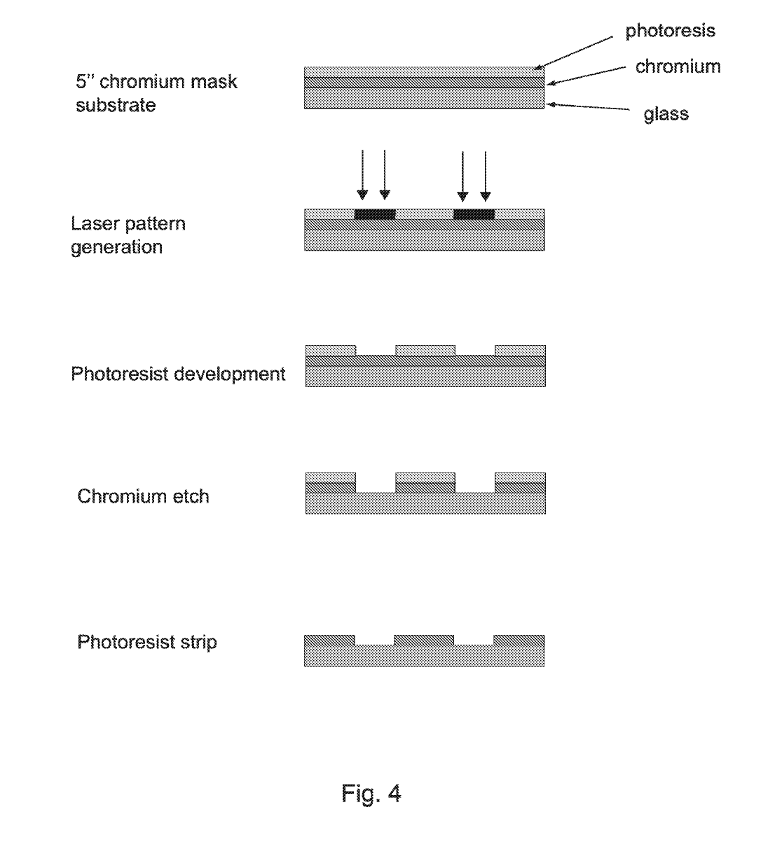

FIG. 3 shows chip fabrication according to the present invention and FIG. 4 shows a mask fabrication process according to the present invention.

We first prepared two masters: one for the flow and another for the control layer. Both masters were printed on chrome plates using standard photolithography techniques.

We then used the chrome masters as masks for fabricating two types of wafers: flow and control. Flow wafers were typically fabricated with AZ9260 positive resist at a thickness of 14 μm (Table 1) and control wafers were fabricated with SU-8 negative photo resist at a thickness of 10 μm (Table 1). Next, we used the fabricated wafers to mold two layers of a PDMS device. Finally, two PDMS layers were aligned and bonded together; we punched holes using a manual-punching machine (Syneo, USA).

### PDMS Dispenser

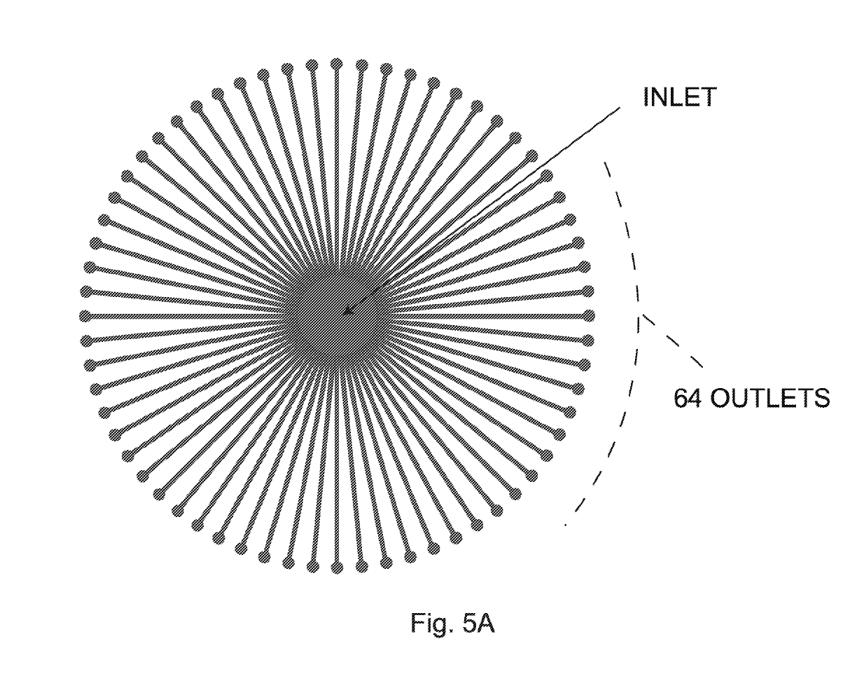

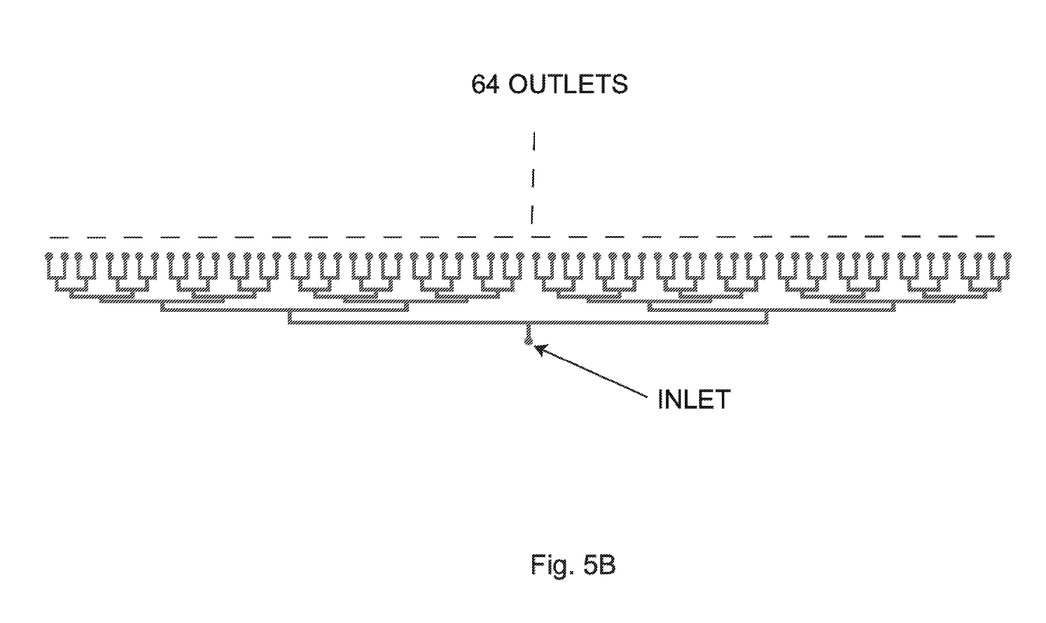

The device, which allows individual access to each working unit, has an advantage of manipulating heterogeneous samples simultaneously without any risk of contamination or crosstalk. Parallel loading of multiple samples on the chip in this case requires several external sources of pressure. The commercially available tools, such as pneumatic manifolds, typically can branch an external source of compressed air creating several daughter sources. Most of the suppliers currently provide pneumatic manifolds made of metal or plastic that can bifurcate the pressure source into 8 to 12 parallel outputs. But for manipulating 64 samples simultaneously one would need to use several manifolds connected in a massive control unit. To avoid this complex construction, save space and facilitate the dispensing of samples into individual chambers we designed and fabricated a “passive” dispenser that is aimed to substitute pneumatic manifold. The aim of this device is to evenly distribute the input compressed air between 64 outlets. Similarly to previously mentioned micro device we fabricated the dispensers using soft lithography. But unlike the two-layer patterned devices, dispenser requires only one molded part that is bonded to non-patterned PDMS. FIGS. 5A and 5B show a design of two PDMS “passive” dispensers, an external source of compressed pressure is connected to the inlet of the device and is equally distributed between 64 outlets.

### Random Nucleotide Library

We exploited the principle of MITOMI-based affinity selection of bound sites from the randomized DNA library to characterize TF DNA binding specificities. We have designed the target DNA library by randomly introducing all four nucleotides at each position of a DNA sequence. This library of random DNA sequences can then be exposed to a TF of interest after which bound sequences are collected and decoded by sequencing. This approach was adopted by several techniques that aim to identify TF binding preferences including HT-SELEX and B1H. Unlike de Bruijn sequences, the design of a random library is relatively simple and synthesis of target oligos is not cost-prohibitive. At the same time, the length of the random site is not limited and one can easily design a library that would cover all possible 10, 20- or even 30-, 40- or 50-mers. This might be especially useful when identifying the binding preferences of homo- and heterodimers. And finally, randomized DNA libraries can be easily multiplexed and used for the characterization of binding specificities of several factors simultaneously.

### MITOMI-Seq: Principle

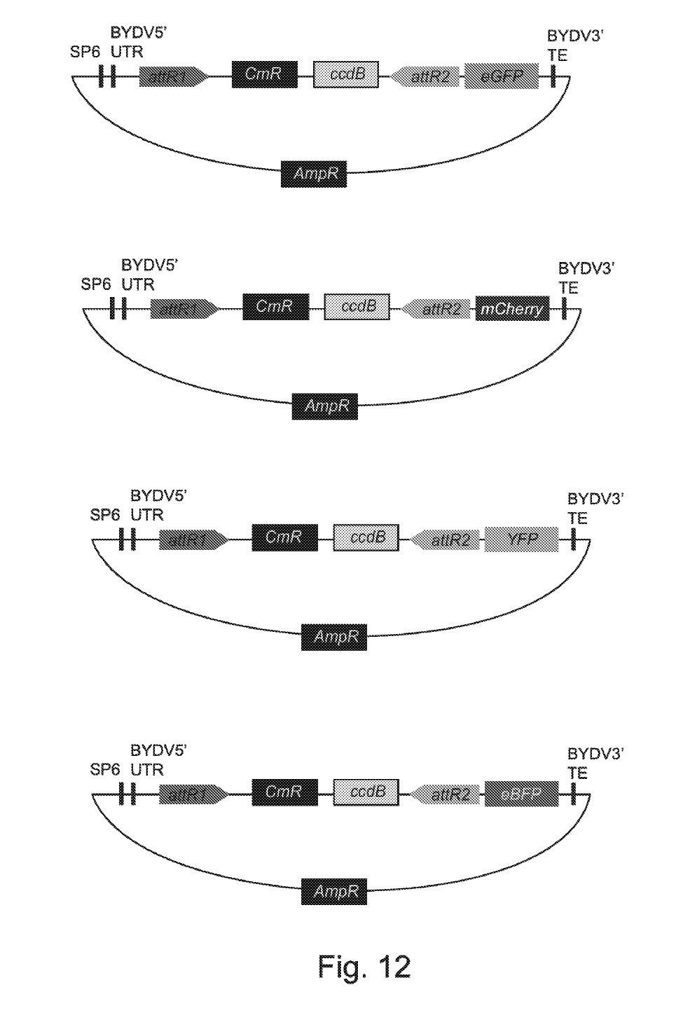

Each MITOMI-seq experiment starts with the in vitro expression of TFs of interest and generation of target DNA libraries. We express TFs of interest in 6 μl of the TnT® SP6 High-Yield Wheat Germ (Promega) protein expression system. To make this expression system compatible with the Gateway cloning format and to allow the fluorescence-based detection of TFs, we shuttle the open-reading frame (ORF) of the TF of interest into one of the custom-made pMARE expression vectors (pMARE-eGFP or pMARE-mCherry (Hens et al., 2011)) (FIG. 12) (for more details, please see Methods section).

The target DNA libraries are constructed from single stranded synthetic oligos by an enzymatic second strand synthesis. Meanwhile, the surface of the microfluidic device is functionalized to capture tagged TFs (the procedure is similar to the one established for regular MITOMI chips, see Methods).

The expressed TFs are then mixed with target random DNA libraries and the mixtures are immediately loaded on the micro device. After 40 minutes of incubation, newly formed TF-DNA complexes are trapped under a flexible “button” membrane, unbound material is removed from the device by washing and bound DNA is collected by continuous elution (for a detailed protocol, please see Methods). Collected DNA is then amplified and sequenced in one lane of HiSeq sequencer (Illumina).

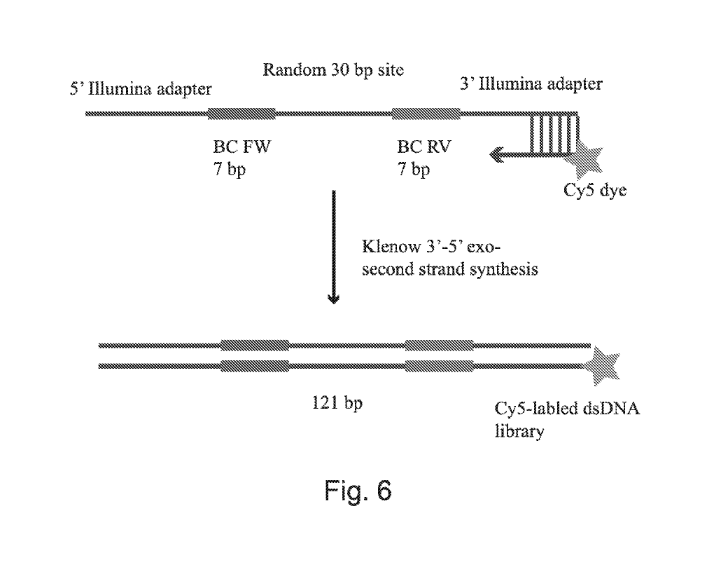

To overcome all the difficulties related to the preparation of samples for HT sequencing, we decided to incorporate sequencing adapters directly within the random DNA library design. To do so we designed target DNA libraries that already contain Solexa (Illumina) sequencing-compatible adapters at the 5′- and 3′-prime ends of the barcoded randomized 30 bp fragment (FIG. 6). The constructs of 121 bp were then synthesized as single-stranded oligos and were later used to generate double-stranded fluorescently labeled target DNA libraries using Klenow 5′-3′ exo-second strand synthesis (For details see Methods).

### MITOMI-Seq: Procedure

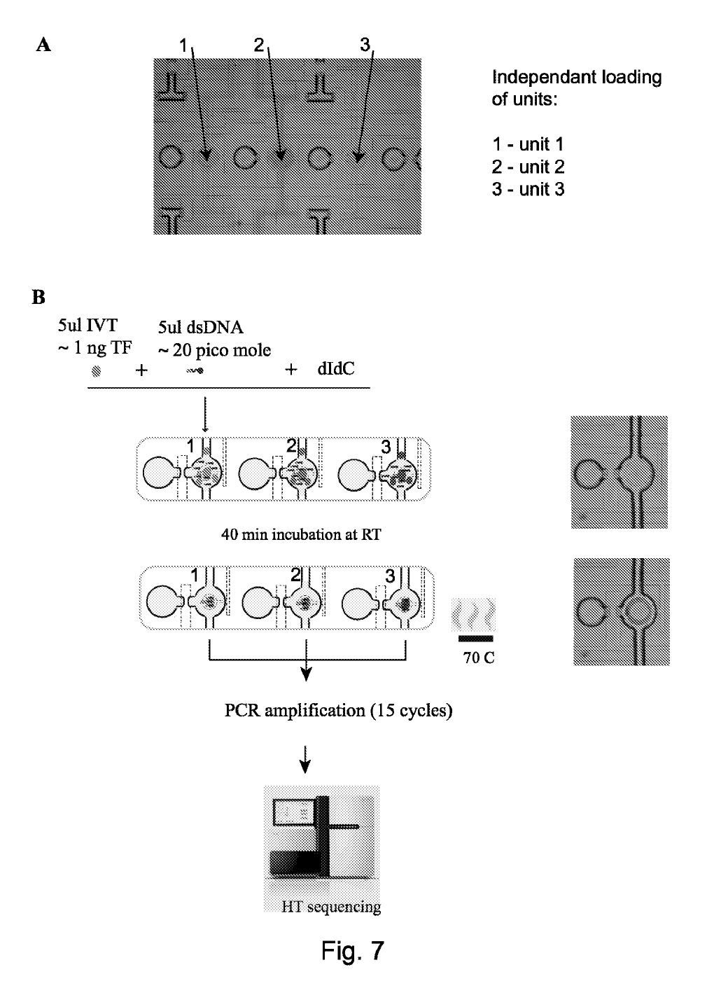

FIG. 7 shows a MITOMI-seq procedure according to the present invention. FIG. 7A shows a snapshot of three arbitrary units of the microfluidic device (unit 1, unit 2 and unit 3), each unit is isolated from others using the microvalves and has an individual inlet and an outlet as illustrated by the independent passage of color dyes.

FIG. 7B shows a MITOMI-seq procedure, Bait TF, target dsDNA and a non-specific competitor poly-dIdC are mixed and loaded in one chamber of the microfluidic device, the mixtures are incubated on-chip for 40 minutes then, bound DNA is eluted from all the units of the device simultaneously and collected in one tube, recovered DNA is amplified and sequenced on a HiSeq instrument.

On the left of FIG. 7B there is shown a schematic representation of three individual chambers, and on the right there is corresponding snapshots of an individual chamber taken before and after mechanical trapping.

For each MITOMI-seq experiment, we mix the DNA libraries with expressed TFs and load these mixtures onto the microfluidic chip (FIG. 7B). After incubating the chip for 40 min at room temperature, we trap the antibody-captured TF-DNA complexes with the button valves and wash the unbound material away. We then also remove non-specifically bound DNA sequences from the immobilized complexes by opening the button for one second under continuous flow of the washing buffer. To elute the remaining (bound) DNA, we resort to heating the microchip to 70° C. for 10 min as explorative experiments revealed that this vastly improved the release of trapped DNA in PBS washing buffer which is applied for 10 min. Recovered DNA is then amplified and sequenced on a HiSeq sequencer (FIG. 7). (For the detailed MITOMI-seq protocol, see Methods).

The resulting sequencing data is then de-multiplexed using the barcodes and reads are trimmed to the random 30 bp fragment that is located between the two barcodes corresponding to each sample. These 30 bp fragments are subsequently sorted according to their frequency: from high to low, and identical reads are collapsed in one. We then extract the top 1500 reads from each sample and use it to generate representative binding motifs with MEME (Bailey and Elkan, 1994).

### Results:

### MITOMI-Seq Identifies DNA Binding Motifs of TF Monomers and Dimers

First we used the extended random library to perform MITOMI-seq on several well-studied TFs for which DNA binding preferences are known. We also included the now well-characterized PPARγ:RXRα heterodimer to the tested set. Indeed, the binding specificity of PPARγ:RXRα heterodimer was previously characterized through ChIP-seq data analysis (Nielsen et al., 2008) but importantly, it has never been probed by any of the high- or medium-throughput in vitro techniques such as PBM, HT-SELEX or B1H.

Motifs obtained by MITOMI-seq for the selected factors closely match those identified previously (Table 2). Interestingly, for PPARγ:RXRα heterodimer, we recovered a motif similar to the one identified by ChIP-seq (Nielsen et al. 2008).

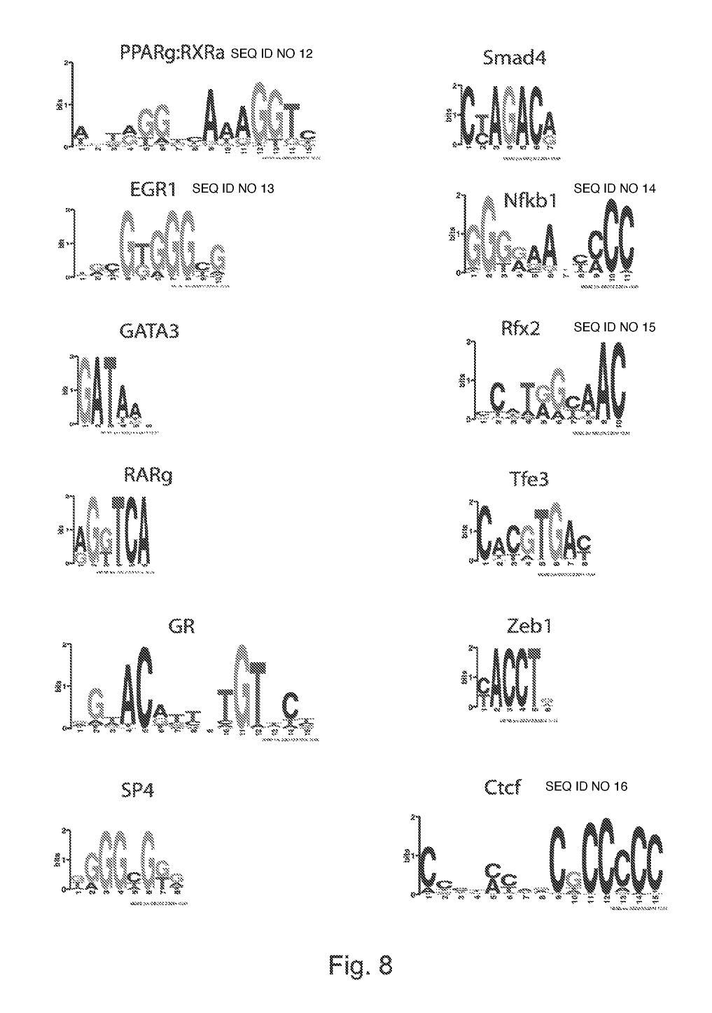

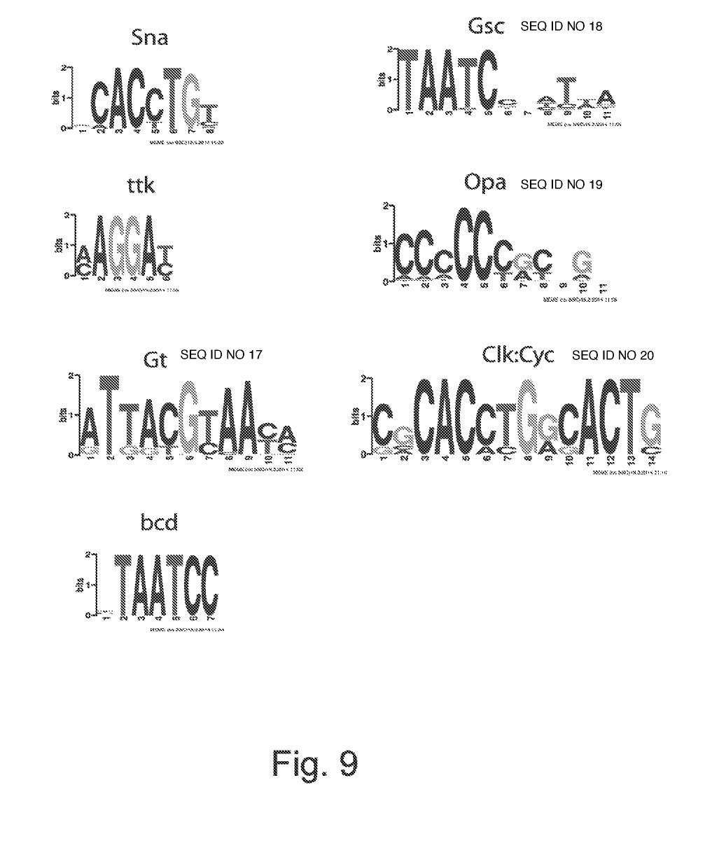

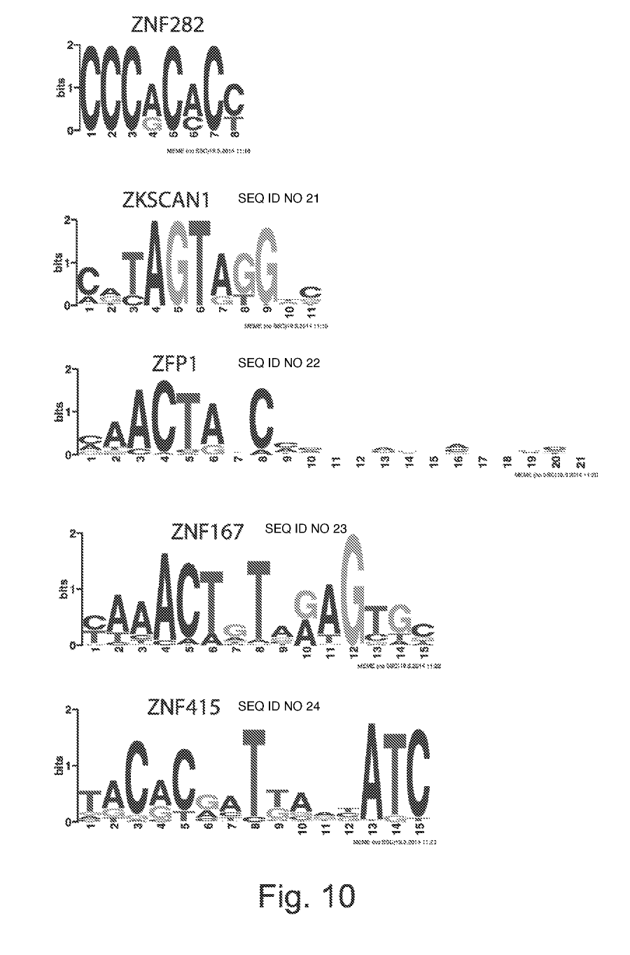

Over a span of just a couple of weeks, we have already been able to process 40 various individual TF or TF dimers originating from different species: M. musculus, D. melanogaster and H. sapiens in three separate MITOMI-seq experiments (24 TFs per experiment). Some factors were processed twice. We were able to retrieve 24 high-confidence motifs with E-values ranging from 1.70E-14 to 4.3E-435 (FIGS. 8, 9, 10). Among those 24 were motifs for TFs that were so far deemed off-limits for DNA binding analyses and for which consequently no binding properties were described. Specifically, we were able to identify motifs for five TFs that belong to the family of KRAB zinc finger proteins: ZNF282, ZKSCAN1, ZFP1, ZNF167 and ZNF415 (FIG. 10). We also identified the binding motif of the zinc finger and homeodomain-containing TF ZEB1 (FIG. 8) and this motif was later confirmed by ChIP-seq analysis (data not shown).

### Comparison of MITOMI-Seq, PBM and HT-SELEX Platforms: NFKB1, a Case Study

We next tried to estimate how well MITOMI-seq performs in comparison to the two other commonly used in vitro technologies that enable TF DNA binding measurements: PBM and HT-SELEX. For this purpose, we focused on NFKB1 DNA binding data sets since data are available from all three technologies. Specifically, we compared the HT-SELEX experimental data from selection cycles 2, 3 and 4 (Dolma et al., 2013), normalized PBM probe data (Siggers et al., 2011) and two MITOMI-seq datasets from independent experiments. Note however that we anticipate including additional comparisons involving other factors in the ultimate manuscript. The NFKB1-related analyses have to therefore be considered as a proof of principle.

First, we asked how well MITOMI-seq-derived PWM models predict HT-SELEX binding models and if there would be a difference in data obtained by the two technologies. We used a representation of an inferred NFKB1 binding model obtained by ChiP-seq in MITOMI-seq or HT-SELEX data as an estimate of the performance of each technique. For five HT-SELEX and MITOMI-seq datasets, we estimated the amount of unique reads obtained after HT sequencing. Next, we used FIMO (Grant et al., 2011) to calculate enrichment of the NFKB1 TRANSFAC motif (ChIP-seq-derived V$NFKB_Q6_01 (Matys et al., 2006)) within unique reads from all five datasets. We found that one MITOMI-seq data set was significantly enriched with sites mapping to the motif (23.70%) as compared to the three datasets obtained by HT-SELEX. A second MITOMI-seq dataset did however not show a similarly high motif enrichment, as the percentage of unique reads that contained the TRANSFAC-derived NFKB1 motif was close to the one found for the 2^{nd} and the 3^{rd} cycle data sets obtained using HT-SELEX. Thus, it appears that the motif enrichment in the data obtained by both technologies is comparable, at least for NFKB1, with MITOMI-seq being potentially in greater agreement with ChIP-seq data than HT-SELEX. We speculate that differences in the enrichment found between two MITOMI-seq data sets could be due to variability in the on-chip selection process between two experiments. We already observed that some experiments result in a wider representation of non-specific DNA binders in sequencing data than others. Nevertheless, we would like to argue that this technical bias does not hinder overall motif discovery.

TABLE 3
NFKB1 motif enrichment in five different datasets generated by MITOMI-seq
or HT-SELEX.

| | | | | The number of | |
|---|---|---|---|---|---|
| | | | | unique reads in | |
| | | | | which | |
| | Total | Unique | | TRANSFAC | Motif |
| NFKB1 data set | reads | reads | Sequencer | motif occurs | enrichment |
| HT-SELEX cycle 2 | 66061 | 65807 | GAII | 1771 | 2.69% |
| HT-SELEX cycle 3* | 440647 | 296195 | HISEQ | 21214 | 7.16% |
| HT-SELEX cycle 4 | 153828 | 115256 | GAII | 11939 | 10.36% |
| MITOMI-seq | 838253 | 291788 | HISEQ | 69167 | 23.70% |
| experiment 1 | | | | | |
| MITOMI-seq | 1033730 | 746221 | HISEQ | 35678 | 4.78% |
| experiment 2 | | | | | |

*Cycle 3 was used to construct the HT-SELEX binding model in the original study.

### Conclusion:

A comprehensive understanding of protein—DNA binding properties is of central importance to gene regulation. Several high-throughput technologies have therefore been developed to study TF-DNA binding. The most popular technology allowing in vivo DNA profiling is ChIP-seq (Johnson et al., 2007). However, it is well appreciated that ChIP-seq-derived DNA binding properties might not provide an accurate picture of TF DNA binding specificities. First, the accessible sites in a particular genome might not cover all possible k-mers. Second, in vivo binding is affected by additional factors, such as chromatin structure, nucleosome positioning and co-factors and thus observed DNA binding in vivo may not even be direct. Thus, in contrast to in vivo studies, in vitro DNA binding assays are valuable because they enable the assessment of direct DNA binding properties, allowing the sampling of the full spectrum of DNA k-mers. In the past, in vitro binding models of TFs were defined based on low-throughput techniques and thus had low resolution and limited accuracy. With technological developments, the ability to measure and predict binding sites has improved. A large leap came in the form of PBMs and HT-SELEX. These two high-throughput technologies produced DNA binding specificity data covering hundreds of TFs. But despite these significant technological advances, all available in vitro binding models taken together currently explain the specificities of only about one third of the total number of known TFs. Moreover, the DNA binding properties of most TF homo- and heterodimers, let alone larger complexes still remains vastly unexplored.

In this study, we aimed to address this problem, presenting a novel platform for the robust characterization of DNA binding specificities of TF monomers and dimers. The platform itself is based on two core technologies: MITOMI and HT-sequencing, which in combination, enable us to examine TF DNA binding from a new perspective and to determine the binding specificities at an unprecedented resolution. MITOMI-seq combines robust selection of sequences bound to a certain TF from a pool of k-mers with subsequent identification of the bound DNA by deep sequencing. As part of these efforts, we also developed an integrative microfluidic device that allows to run several MITOMI-seq assays simultaneously and to process 64 TFs or TF combinations in parallel. The device is based on the MITOMI principle and performs physical trapping of TF-DNA complexes thereby reducing the loss of bound DNA during the washing step to a minimum. This, in turn, illuminates the unique property of the assay, namely, the ability to preserve and analyze interactions over a wide affinity range. Unlike PBM and HT-SELEX, the two most popular in vitro technologies that aim to identify TF binding specificities, MITOMI-seq operates at micro scale and requires minute amounts of biological material. For example, to perform MITOMI-seq on one TF, one needs only few nanograms of protein which can be easily produced through available in vitro expression systems. Thus, tedious bacterial or mammalian protein expression and purification is no longer required which significantly shortens the time needed for one experiment (Table 4) and even allows the analysis of TFs (such as KRAB ZFPs) that are otherwise difficult to study because of in cello expression issues.

We demonstrated that MITOMI-seq-derived specificity models generally agree with the TF binding models identified by ChIP-seq or by available in vitro methods (Table 2). We also showed that MITOMI-seq could identify DNA binding preferences of not only monomers or homodimers but also—of TF heterodimers. Particularly, using MITOMI-seq data, we were able to generate relevant binding models for PPARγ:RXRα and Clk:Cyc heterodimers through de novo motif discovery. In addition, we identified for several factors binding motifs that were never reported before. Two identified motifs (for ZEB1 and ZNF282) were later confirmed by ChIP-seq.

To understand the potential of MITOMI-seq data and how similar its performance is to PBM or HT-SELEX data, we also compared the output of MITOMI-seq to that of the other two in vitro technologies. A close comparison of NFKB1 datasets generated from HT-SELEX and MITOMI-seq revealed that unlike SELEX, MITOMI does not result in an over-selection of the same DNA sequences but instead provides a larger fraction of informative sites that contribute to a specificity model. Thus, binding models generated from MITOMI-seq data may potentially be more accurate and comprehensive compared to HT-SELEX models. At the same time, we showed that, similar to observations with the PBM approach, the DNA binding data from MITOMI-seq can guide DNA binding affinity estimates. We also showed that the sequences that were enriched in the NFKB1 MITOMI-seq data set were also ranked as highest NFKB1 affinity sites in the PBM data. This suggests that, in principle, MITOMI-seq and PBM can produce comparable data. But unlike PBM, MITOMI-seq allows the probing of a much larger sequence space making it an ideal platform for the identification of sites bound by TF dimers or by factors that recognize long DNA sequences.

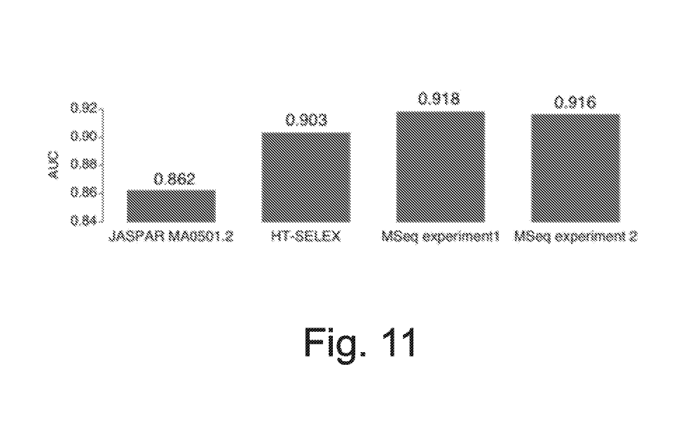

One common concern about the DNA binding data obtained by various in vitro technologies, including MITOMI-seq, is how relevant in vitro models are to in vivo binding and if there are certain advantages or disadvantages of each in vitro technology with respect to one another. Currently we are investigating this problem and trying to assess on how well can MITOMI-seq data predict in vivo TF DNA binding in general and if there any advantage of it compared to PBM and HT-SELEX. As a first attempt to evaluate the “goodness” of the motifs derived by MITOMI-seq, HT-SELEX and the motif retrieved from JASPAR database (ChIP-seq based) we quantified the occurrence of each of the motifs within the ChIP-seq peaks corresponding to NFKB1 binding in lymphoblastoid cells. We used sensitivity at 1% false-positive and area under the receiver operating characteristic curve to gauge the binding prediction (see Orenstein and Shamir, 2014 for details). We found the area under curve (AUC) value to be higher for MITOMI-seq-derived motif compared to HT-SELEX and JASPAR motifs (FIG. 11. JASPAR, HT-SELEX and MITOMI-seq motif occurrence in ChIP-seq peaks of NFKB1 derived from human lymphoblastoid cell line (GEO:GSM935527). This proves that in the case of NFKB1 binding the motif derived by MITOMI-seq is more accurate in predicting binding sites of NFKB1 genome-wide compared to previously available binding models.

TABLE 4
Comparison of PBM, SELEX-seq and MITOMI-seq technologies.

| | PBM | | |
|---|---|---|---|
| | (Protein binding | | |
| Property | microarrays) | SELEX-seq | MITOMI-seq |
| Sequence space | limited | large | large |
| Throughput | low | high | high |
| Nature of the | medium-to-high | high | low-to-high |
| interactions | affinity | affinity | affinity |
| Protein | yes | yes | no |
| purification | | | |
| needed | | | |
| Ability to monitor | yes | no | yes |
| heterodimers | | | |
| Amount of protein | μg | ng | ng |
| required | | | |
| Hands-on | ~2 days | ~2 days | 4-5 hours |
| procedure | | | |
| Need for the | yes | no | no |
| preprinted | | | |
| microarray | | | |

### Methods:

### Protein Cloning and Expression

To enable the expression of TFs and their immobilization and fluorescence-based detection, we explored different strategies. We found that the wheat germ (WG) in vitro transcription translation expression system containing translation enhancer (TE) sequences from the barley yellow dwarf virus (BYDV) (Promega) yielded the most robust and reproducible protein expression (data not shown). To make this expression system compatible with Gateway TF ORF clone format and to allow the fluorescence-based detection of TFs, we generated several novel vectors, pMARE, that differ by fluoro C-terminal fusions (FIG. 12). To do so we cut the pF3A WG (BYDV) Flexi vector (Promega) with NcoI and DraI, and removed the barnase cassette. The NcoI site was blunted and the Gateway reading frame A cassette (Invitrogen) was ligated in. Subsequently, the eGFP, mCherry, YFP of eBFP coding sequences containing a stop codon at the 3′-end was incorporated between the KpnI and Sac/restriction sites using standard cloning techniques. TFs were then subcloned from the Entry clones into the pMARE vector by standard Gateway cloning.

### Target DNA Library Preparation

Randomized extended DNA libraries were ordered as single stranded oligos from IDT. The adapter sequences and barcodes used for each library are listed in the Table 5. The oligo containing a Cy5 5′-fusion: /5Cy5/CAA GCA GAA GAC GGC ATA CG (SEQ ID NO 9) was used as a primer of the complementary strand synthesis by means of Klenow exo-extension reaction (NEB Cat No M0212). Detailed reaction conditions are described in the MITOMI-seq procedure. The libraries were then purified using MinElute PCR purification kit (Qiagen) and diluted with ddH20 in a ration 1:10. 50 ng of poly-dIdC (Sigma) were added to each 10 μl of the diluted library.

### MITOMI-Seq Procedure (Detailed Protocol):

1. Sample Preparation:

1.1. Set Up the Expression Mix for the TFs as Follows:
4 WITT mix (TnT® SP6 High-Yield Wheat Germ master mix, Promega)
˜200 ng plasmid DNA
Nuclease-free dH2O till 6 μl total volume
Incubate at 25° C. for 3 hours

1.2. Synthesis of the dsDNA Libraries:
5 μl Buffer 2 (NEB)
5 μl dNTPs
0.5 μl Cy5 labeling primer (500 μM)
1.5 μl Library oligoes (200 μM)
37 μl dH2O
94° C.-5 min
50° C.-60 sec
place tubes on ice
add 1 μl of Klenow 3′-5′ exo-(NEB Cat No M0212)
37° C. —60 min
keep at 0° C.
   Use MinElute to purify the double-stranded libraries, elute in 12 μl of EB.
   Dilute the libraries 1:10 in ddH2O and add 50 ng of dIdC to each 10 μl of diluted libraries.

2. MITOMI:

2.1. Surface Chemistry:
Load all control lines with dH2O, at ˜3 psi, check that all valves work: switch all ON that the air pushes water through the pipes
   Usually “Button” valves are loading earlier, switch it off once it is filled.
   Increase the pressure in valves till ˜10 psi and make sure that that valves work properly.
Run BSA-biotin for 10 min 2 mg/ml BSA-bio @ 5 psi (keep chambers isolated)
Wash with PBS for ˜2 minutes
Run Neutravidin (500 ug/ml in PBS) for 10 minutes
PBS for ˜2 minutes
Close button
PBS for 1-2 min
BSA-biotin for 10 min 2 mg/ml BSA-bio @ 5 psi (keep chambers isolated)
PBS for 3-4 min
Anti-GFP-biotin 3 min (1:100) (keep chambers isolated)
Open button
Continue Anti-GFP-biotin for 15 min (1:100) (keep chambers isolated)
PBS for 5 min

2.2. Sample Loading and MITOMI:
Mix:
   5 μl expressed non-purified TF
   5 μl diluted ds DNA library
   (optional) 5 μl of a partner TF
Load the mixtures through individual inlets
Incubate at RT for 40 min
Scan with a fluorescent scanner
Close the button
Wash with PBS for 20 min
Scan with a fluorescent scanner

3. Elution and Library Preparation for HT-Sequencing
Open the button
Incubate 70° C. for 10 min
Elute with PBS for 10 min @ 70° C. and amplify as follows:

| | |
|---|---|
| 5xHF KAPA buffer | 10 μL |
| dNTPs (supplied with the kit) | 1.5 μL |
| primer GA2seq FW (10 μM) | 0.5 μL |
| primer GA2seq RV(10 μM) | 0.5 μL |
| KAPA HiFi polymerase | 0.5 μL |
| Eluted DNA | 37 μL |
| Total volume | 50 μL |

Cycle:
95° C. —2 min
98° C. —20 sec|
65° C. —15 sec|×15
72° C. —90 sec|
72° C. —2 min
4° C. —forever
PCR purify using Minelute kit from QIAGEN. Elute in 10 μL of EB.

The detailed information about primers, barcodes and libraries used in this study could be found in a supplementary TableS1.

### Data Analysis

Raw Illumina reads were processed using custom perl scripts, FASTX-tools. Read statistics and HMM were implemented using custom scripts. De novo motif discovery was done with MEME.

TABLE 5
List of barcodes and adapter sequences used
for extended library.

| Sample | barcode FW | barcode RV |
|---|---|---|
| R30_B1 | CATGCTC | GAGCATG |
| R30_B2 | ACGCAAC | GTTGCGT |
| R30_B3 | TCGCAGG | CCTGCGA |
| R30_B4 | CTCTGCA | TGCAGAG |
| R30_B5 | CCTAGGT | ACCTAGG |
| R30_B6 | GGATCAA | TTGATCC |
| R30_B7 | GCAAGAT | ATCTTGC |
| R30_B8 | ATGGAGA | TCTCCAT |
| R30_B9 | CTCGATG | CATCGAG |
| R30_B10 | GCTCGAA | TTCGAGC |
| R30_B11 | ACCAACT | AGTTGGT |
| R30_B12 | CCGGTAC | GTACCGG |
| R30_B13 | AACTCCG | CGGAGTT |
| R30_B14 | TTGAAGT | ACTTCAA |
| R30_B15 | ACTATCA | TGATAGT |
| R30_B16 | TTGGATC | GATCCAA |
| R30_B17 | CGACCTG | CAGGTCG |
| R30_B18 | TAATGCG | CGCATTA |
| R30_B19 | AGGTACC | GGTACCT |
| R30_B20 | TGCGTCC | GGACGCA |
| R30_B21 | GAATCTC | GAGATTC |
| R30_B22 | GCATTGG | CCAATGC |
| R30_B23 | TGACGTC | GACGTCA |
| R30_B24 | GATGCCA | TGGCATC |

| | |
|---|---|
| Cy5 primer | CAA GCA GAA GAC GGC ATA CG |
| | SEQ ID NO 9 |
| Adaptor FW | ACACTCTTTCCCTACACGACGCTCTTCCGATCT |
| (1.b) | SEQ ID NO 10 |
| Adaptor RV | GATCGGAAGAGCTCGTATGCCGTCTTCTGCTTG |
| (1.a) | SEQ ID NO 11 |

### BIBLIOGRAPHY

Bailey, T. L., and Elkan, C. (1994). Fitting a mixture model by expectation maximization to discover motifs in biopolymers. Proc. Int. Conf. Intell. Syst. Mol. Biol. ISMB Int. Conf. Intell. Syst. Mol. Biol. 2, 28-36.
Garcia-Cordero, J. L., and Maerkl, S. J. (2014). A 1024-sample serum analyzer chip for cancer diagnostics. Lab. Chip.
Geertz, M., Shore, D., and Maerkl, S. J. (2012). Massively parallel measurements of molecular interaction kinetics on a microfluidic platform. Proc. Natl. Acad. Sci. U.S.A. 109, 16540-16545.
Grant, C. E., Bailey, T. L., and Noble, W. S. (2011). FIMO: scanning for occurrences of a given motif. Bioinformatics 27, 1017-1018.
Hens, K., Feuz, J.-D., Isakova, A., Iagovitina, A., Massouras, A., Bryois, J., Callaerts, P., Celniker, S. E., and Deplancke, B. (2011). Automated protein-DNA interaction screening of Drosophila regulatory elements. Nat. Methods 8, 1065-1070.
Hong, J. W., and Quake, S. R. (2003). Integrated nanoliter systems. Nat. Biotechnol. 21, 1179-1183.
Johnson, D. S., Mortazavi, A., Myers, R. M., and Wold, B. (2007). Genome-Wide Mapping of in Vivo Protein-DNA Interactions. Science 316, 1497-1502.
Jolma, A., Yan, J., Whitington, T., Toivonen, J., Nitta, K. R., Rastas, P., Morgunova, E., Enge, M., Taipale, M., Wei, G., et al. (2013). DNA-Binding Specificities of Human Transcription Factors. Cell 152, 327-339.
Laser, D. J., and Santiago, J. G. (2004). A review of micropumps. J. Micromechanics Microengineering 14, R35-R64.
Maerkl, S. J., and Quake, S. R. (2007). A systems approach to measuring the binding energy landscapes of transcription factors. Science 315, 233-237.
Matys, V., Kel-Margoulis, O. V., Fricke, E., Liebich, I., Land, S., Barre-Dirrie, A., Reuter, I., Chekmenev, D., Krull, M., Hornischer, K., et al. (2006). TRANSFAC and its module TRANSCompel: transcriptional gene regulation in eukaryotes. Nucleic Acids Res. 34, D108-110.
Nielsen, R., Pedersen, T. A., Hagenbeek, D., Moulos, P., Siersbaek, R., Megens, E., Denissov, S., Børgesen, M., Francoijs, K.-J., Mandrup, S., et al. (2008). Genome-wide profiling of PPARgamma:RXR and RNA polymerase II occupancy reveals temporal activation of distinct metabolic pathways and changes in RXR dimer composition during adipogenesis. Genes Dev. 22, 2953-2967.
Orenstein, Y., and Shamir, R. (2014). A comparative analysis of transcription factor binding models learned from PBM, HT-SELEX and ChIP data. Nucleic Acids Res.
Siggers, T., Chang, A. B., Teixeira, A., Wong, D., Williams, K. J., Ahmed, B., Ragoussis, J., Udalova, I. A., Smale, S. T., and Bulyk, M. L. (2011). Principles of dimer-specific gene regulation revealed by a comprehensive characterization of NF-κB family DNA binding. Nat. Immunol. 13, 95-102.
Unger, M. A. (2000). Monolithic Microfabricated Valves and Pumps by Multilayer Soft Lithography. Science 288, 113-116.
Weibel, D. B., Kruithof, M., Potenta, S., Sia, S. K., Lee, A., and Whitesides, G. M. (2005). Torque-actuated valves for microfluidics. Anal. Chem. 77, 4726-4733.

1-24. (canceled)

25. A microfluidic device for mechanically induced trapping of molecular interactions including a first unit cell and a second unit cell, each unit cell comprising:
a membrane chamber having a membrane;
a flow channel crossing the membrane chamber and having an inlet and an outlet; and
the flow channel crossing the first unit cell being other than the flow channel crossing the second unit cell, or being not the same as the flow channel crossing the second unit cell.

26. The microfluidic device according to claim 25, further comprising:
a first set of components configured to provide an independent fluidic communication to each unit cell.

27. The microfluidic device according to claim 26, further comprising:
a second set of components configured to connect at least the first and the second unit cells together.

28. The microfluidic device according to claim 27, wherein the first set of components or the second set of components include control valves.

29. The microfluidic device according to claim 25, wherein a surface of the microfluidic device is functionalized to capture proteins.

30. The microfluidic device according to claim 29, wherein the proteins include at least one of transcription factors and other biomedically important proteins.

31. A dispenser for parallel loading of multiple samples on a microfluidic device according to claim 25, comprising one inlet and a plurality of outlets to equally distribute the samples, wherein the number of outlets corresponds to the number of unit cells of the microfluidic device.

32. The dispenser according to claim 31, wherein the dispenser is made of polydimethylsiloxane (PDMS).

33. The dispenser according to claim 31, wherein the inlet is directly connected to each outlet.

34. The dispenser according to claim 31, wherein the inlet is connected to the outlets via several channels.

35. A method for isolation of specifically bound nucleic acids to target molecules comprising:
providing a microfluidic device according to claim 25;
loading a mixture of nucleic acids and target molecules into the microfluidic device;
trapping the bound nucleic acid-target molecule complexes;
removing the unbound material;
collecting the bound nucleic acids;
amplifying the bound nucleic acids; and
high throughput sequencing of the amplified bound nucleic acids.

36. The method according to claim 35, wherein target molecules include proteins.

37. The method according to claim 36, wherein the proteins are expressed proteins.

38. The method according to claim 35, wherein the proteins include single or multiple proteins.

39. The method according to claim 35, wherein the proteins include transcription factors (TFs) or other biomedically important proteins.

40. The method according to claim 35, wherein the nucleic acids include a random DNA library.

41. The method according to claim 40, wherein the random DNA library include sequencing adapters.

42. The method according to claim 35, wherein the nucleic acids include a random RNA library.

43. The method according to claim 35, further comprising the step of:
loading the mixture of nucleic acids and target molecules on the membrane chamber of the microfluidic device.

44. The method according to claim 35, wherein bound nucleic acids are collected at the same time.

45. The method according to claim 35, wherein bound nucleic acids are collected while heating the microfluidic device.
